(19) United States
(12) Patent Application Publication
(10) Pub. No.: US 2025/0195528 A1
(43) Pub. Date: Jun. 19, 2025

(54) DOSE REGIMEN OF VICASINABIN

(71) Applicant: Hoffmann-La Roche Inc., Little Falls, NJ (US)

(72) Inventors: Katrijn Bogman, Folgensbourg (FR); Antonello Luigi Girolamo Caruso, Rheinfelden (CH); Barbara Ecabert, Therwil (CH); Andreas Guenther, Steinen (DE); Ulrich Friedrich Oskar Luhmann, Riehen (CH); Agnès Poirier, Basel (CH); Bernhard Steiert, Weil am Rhein (DE); Christoph Ullmer, Fischingen (DE)

(73) Assignee: Hoffmann-La Roche Inc., Little Falls, NJ (US)

(21) Appl. No.: 19/065,116

(22) Filed: Feb. 27, 2025

Related U.S. Application Data
(63) Continuation of application No. PCT/EP2023/073741, filed on Aug. 30, 2023.

(30) Foreign Application Priority Data
Sep. 1, 2022 (EP) 22193362.5
May 15, 2023 (EP) 23173373.4

(51) Int. Cl. A61K 31/519; A61K 45/06

(52) U.S. Cl. CPC A61K 31/519; A61K 45/06

(57) ABSTRACT

The present invention relates to (3S)-1-[5-tert-butyl-3-[(1-methyltetrazol-5-yl)methyl]triazolo[4,5-d]pyrimidin-7-yl]pyrrolidin-3-ol for use in the treatment of diabetic retinopathy, and its methods of treatment thereof.

### CROSS-REFERENCE TO RELATED APPLICATIONS

This application is a continuation of PCT Application No. PCT/EP2023/073741, filed Aug. 30, 2023, which claims benefit of priority to EP Application No. 22193362.5, filed Sep. 1, 2022 and EP Application No. 23173373.4, filed May 15, 2023, each of which is incorporated herein by reference in its entirety.

### BACKGROUND

Diabetes mellitus affects 463 million people worldwide and its prevalence is expected to grow to 700 million by 2045 (IDF 2019; Nair et al. 2016). Diabetic retinopathy (DR) affects approximately one-third of Type 1 and Type 2 diabetic patients (Yau et al. 2012). Chronic hyperglycemia causes retinal microvascular alterations, inflammation, and neurodegeneration (Fong et al. 2004). At any stage of DR, patients may develop diabetic macular edema (DME), the most common cause of central vision loss in patients with diabetes (Resnikoff et al. 2004). DME, together with the advanced stages of DR (i.e., proliferative diabetic retinopathy [PDR]) impairs vision and can lead to blindness (Leasher et al. 2016). Overall, DR is estimated to be the most frequent cause of new cases of blindness among working-aged adults around the world (American Diabetes Association 2002; Yau et al. 2012).

Unlike in other parts of the world, patients in the US with DR can be treated by intravitreal (IVT) injection of anti-vascular endothelial growth factor (VEGF) regardless of the disease severity or presence of visual impairment, PDR, or DME (Lucentis USPI; Eylea USPI). However, clinical practice trends indicate that a high proportion of patients with non-proliferative diabetic retinopathy (NPDR) and with DME resulting in minimal visual impairment are not readily receiving treatment (Cantrell et al. 2020). Instead, treatment initiation is deferred until the disease progresses and further damage to the retinal anatomy and worsening of vision occur. While patients with severe NPDR have no distinct visual impairment yet, their underlying retinal pathology is progressive. Moderately severe to severe NPDR, left untreated, may progress to vision-threatening disease in about 41% and 58% of patients within 1 and 2 years, respectively (Regeneron Pharmaceuticals, Inc. 2020). Thus, there remains an unmet need for the development of novel therapies that provide non-invasive treatment options of severe NPDR and prevent progression to visual impairment.

Chronic hyperglycemia can cause DR or DME through pathological mechanisms that are not fully understood but appear to involve four major pathways: the polyol pathway, advanced glycation end products pathway, protein kinase C pathway, and hexosamine pathway (Brownlee 2005). All of these pathways lead to increased oxidative stress, inflammation, vascular dysfunction, and neurodegeneration (Fong et al. 2004). Müller cells, microglia, and endothelial cells produce chemokines that induce leukostasis, diapedesis, influx of systemic immune cells such as monocytes into the retina, and increased production of cytokines including VEGF, tumor necrosis factor-α, interleukin (IL)-1/p, IL-6, metalloproteinases, and angiopoietin-2. These inflammatory mediators then result in the breakdown of endothelial cell-cell junctions forming the blood-retinal barrier. Retinal microglia become activated at early stages of the disease process, and there is increased apoptosis of ganglion cells and amacrine cells. In retinal capillaries, pericyte dropout and thickening of the basement membrane also occur as a result of hyperglycemia, all contributing to increased leakage from vessels. The above factors drive vascular pathology ultimately causing loss of vision (Das et al. 2015).

DR may be very broadly classified into two stages based on the level of microvascular degeneration and related ischemic damage: (a) NPDR, characterized by microaneurysms and possible intraretinal hemorrhage, hard exudates and cotton wool spots, and (b) PDR, whose hallmarks include neovascularization of the retina, neovascularization of the iris (NVI), and vitreous hemorrhage (Wilkinson et al. 2003). Another complication of DR is DME, which can occur across all DR severity levels and represents the most common cause of vision loss in patients with DR. DME arises from diabetes-induced breakdown of the blood-retinal barrier, with consequent vascular leakage of fluid and circulating proteins into the neural retina. This leads to abnormal retinal thickening and often cystoid edema of the macula (Frey and Antonetti 2011; Zhang et al. 2014; Stitt et al. 2016). Hyperglycemic conditions also lead to retinal ischemia, which induces increased VEGF production that is responsible for vascular leakage, neovascularization, and PDR.

### Current Therapies and Unmet Medical Need

Intraocular treatment modalities for diabetic eye disease include laser photocoagulation, and IVT of anti-VEGF or steroid agents. Current therapeutic paradigms comprising laser photocoagulation and/or IVT anti-VEGF focus on the treatment of advanced disease, once PDR or DME have developed. Anti-VEGF agents are approved for the treatment of all forms of DR in the United States (Lucentis USPI; Eylea® USPI) and for the treatment of DME and PDR elsewhere (Lucentis SmPC) and constitute the first-line therapy for most eyes with center-involving DME. IVT injections of steroid can also be effective for DME treatment (Campochiaro et al. 2012; Boyer et al. 2014); however, IVT steroid use is usually limited by more frequent ocular side effects, such as cataract and glaucoma. According to the 2017 guidelines for diabetic eye care from the International Council of Ophthalmology, NPDR warrants close monitoring, and laser photocoagulation can be considered; however use of anti-VEGF is not mentioned (Wong et al. 2018). Despite the broad label of anti-VEGF therapies for DR in the US (Lucentis USPI; Eylea® USPI), clinical practice trends indicate that a high proportion of patients with NPDR with or without DME causing minimal visual impairment are not readily receiving treatment (PAT Survey; Cantrell et al. 2020) and treatment initiation is instead deferred until the disease progresses and further damage to the retinal anatomy and worsening of vision occur. Several factors may drive the deferral of treatment for moderately severe to severe NPDR in current practice and could potentially be addressed with oral RO6868847. These include:
Invasiveness of available therapies: All currently available ocular therapies for DR/DME (e.g., laser, IVT injections) require invasive procedures and are associated with infrequent but serious risks. In addition, patients report feelings of anxiety regarding initiation of the IVT injection procedure (Senra et al. 2017). The Sponsor has developed RO6868847 for oral administration, thus offering a non-invasive alternative to the IVT injection procedure and associated risks, to improve patient experience, especially considering that DM is a systemic condition and DR is usually bilateral.
High treatment/visit frequency of IVT therapy: Initiation of anti-VEGF therapy for DR/DME, at least in the first year, implies a high frequency of treatments and/or follow-up visits in order to establish and maintain benefits (Lally et al. 2016). As an oral drug, RO6868847 can be continuously self-administered, and its effects are not subjected to IVT drug application and elimination. Because of this, it is envisioned that the interval between follow-up visits will be longer with RO6868847 than for IVT anti-VEGF therapies, which will reduce burden on patients and healthcare systems.
Low perceived urgency to treat: Compared with neovascular age-related macular degeneration (AMD), DR has a slower disease progression and is perceived to be relatively forgiving, and retinal damage associated with disease progression is incorrectly considered to be largely reversible. An extrapolation from pivotal trials with anti-VEGF therapy for DME (Brown et al. 2013; Schmidt-Erfurth et al. 2014; Heier et al. 2016) indicates that although deferred therapy may lead to a reduction in retinal thickness (structural benefit) comparable to immediate therapy, there seems to be a reduced potential for visual recovery (functional benefit) with deferred anti-VEGF therapy. There is consensus that earlier detection and treatment of DR/DME could minimize or prevent both disease progression and the risk of vision loss (Nair et al. 2016).
Lack of consensus on how to treat: In contrast to DME, there is no clear consensus about management of patients with DR and good vision (BCVA>73 letters) with anti-VEGF therapy, as shown by the 2018 PAT Survey (Singh 2018). New evidence from the PANORAMA trial (Regeneron Pharmaceuticals, Inc. 2020) has recently been published, suggesting a treatment protocol of every two months injection (after 5 monthly treatments) for aflibercept to improve DR. In addition, the clinical trial Protocol W from the Diabetic Retinopathy Clinical Research Network (Protocol W) demonstrated that anti-VEGF therapy can prevent the development of sight-threatening complications in eyes with high risk of PDR.

While patients with moderately severe to severe NPDR have no distinct visual impairment, their underlying retinal pathology is progressive (Morello 2007). Moderately severe to severe NPDR, if left untreated, may progress to vision-threatening disease in approximately 41% of patients within 1 year and 58% of patients in two years (Regeneron Pharmaceuticals, Inc. 2020). Thus, there remains an unmet need for the development of novel therapies that provide non-invasive treatment options for earlier intervention and before irreversible retinal damage occurs.

In summary, patients presenting with NPDR in clinical practice are often not immediately treated with IVT anti-VEGF therapy for a number of possible reasons. Deferral of DR therapy in these patients is likely disadvantageous as this is a progressive disease. Many of the given reasons to defer treatment with IVT anti-VEGF therapy may be potentially addressed by RO6868847, an oral small-molecule agonist highly specific for the cannabinoid receptor 2 (CB2). An oral small-molecule agonist, such as RO6868847 (also referred to as RG7774 or vicasinabin), may be appropriate for the treatment of patients with all stages of NPDR and earlier stages of advanced complications of DR (PDR, DME), for which IVT anti-VEGF injections are either not approved/reimbursed or not administered in clinical practice. DR is frequently bilateral, and based on its intended oral route, RO6868847 may benefit both eyes simultaneously. Many studies have shown that cannabinoids can suppress the production of cytokines in innate and adaptive immune responses, both in animal models and in human cell cultures (Klein 2005). The suppression of pro-inflammatory cytokine and chemokine production indicates that synthetic CB2 agonists may have anti-inflammatory effects and could therefore be used for the treatment of chronic inflammatory diseases such as DR.

RO6868847 is a white to off-white powder that shows moderate to good solubility in organic solvents and is slightly soluble in aqueous solutions. RO6868847 is fairly stable to temperature stress and stable to light in solid state and in solution. For Phase I, film-coated tablets for oral administration containing 0.05, 0.75, 10, or 100 mg of RO6868847 drug substance were developed for clinical use. Three placebo film-coated tablets were formulated to match the active film-coated tablets in size and in appearance: one size to match the active 0.05 and 0.75 mg film-coated tablets and one size each to match the active 10 and 100 mg film-coated tablets.

### Rationale for RO6868847 Treatment

RO6868847 is a highly selective CB2 agonist intended for the oral treatment of all stages of NPDR and earlier stages of advanced complications of DR (PDR, DME). CB2 is mainly expressed in immune cells, including microglia in the retina. In the eye, activation of CB2 by RO6868847 has been shown to produce anti-inflammatory effects by inhibiting leukocyte adhesion and microglia activation, decreasing vascular permeability, and consequently preserving endothelial barrier function. Moreover, RO6868847 is highly selective for CB2, thereby excluding psychotropic effects of central CB1 activation (Topol et al. 2010). Current data suggest that RO6868847 displays the highest selectivity compared with publicly known CB2 agonists (Soethoudt et al. 2017). Oral dosing could benefit patients with DR by achieving systemic and bilateral retinal exposure. Based on preclinical evidence, systemic exposure may target leukocytes to prevent attachment to retinal endothelial cells, while retinal exposure may activate the CB2 receptors in microglia and preserve them in a non-activated ramified state. Furthermore, preclinical evidence implies that systemic exposure may have benefits for other diabetic complications, such as diabetic nephropathy, which was shown to be ameliorated in the same nonclinical models as those used for the retinal microglia analysis (Zoja et al. 2016). In summary, due to the advantages of this non-invasive oral administration and the expected bilateral benefits, physicians may more readily start treating all patients with DR who in current practice may only receive deferred IVT therapy. In addition, use of a highly selective CB2 agonist is expected to be devoid of CB1 modulation and respective side effects. The development program of RO6868847 was initiated in order to offer a treatment option for patients with moderately severe to severe NPDR, for whom photocoagulation and intravitreal anti-VEGF therapy is commonly deferred in current practice.

A key challenge for selecting the dose range for the clinical development of RO6868847 was the broad predicted pharmacological active dose range in human lying between doses of 0.22 and 42 mg per day based on different non-clinical animal models. In addition to the non-clinical in vivo data, CB2 receptor target engagement (TE) information from human whole blood was used to select the clinical dose range. Since monitoring CB2R-TE on microglia cells in the eye in vivo is not feasible, CB2R-TE by RO6868847 has instead been predicted using observed plasma PK in humans combined with derived affinity data based on a peripheral ex vivo CB2R-TE on B cells, a circulating immune cell type that expresses CB2 receptors. The CB2R-TE in the retina was estimated assuming that plasma concentrations of RO6868847 are reflective of concentrations in the retina, and that binding affinity of RO6868847 to the CB2 receptor on B cells as assessed in human blood using an ex vivo target engagement assay is representative of binding to the CB2 receptor in the retina. Surprisingly, in vitro experiments to assess CB2R-TE in whole blood samples from elderly and age- and demographically matched diabetic and non-diabetics revealed that roughly a 3-fold higher concentration of RO6868847 is required to achieve a similar effect of CB2R-target engagement as observed in blood of younger healthy participants.

In the Phase I single ascending dose (SAD) and multiple ascending dose (MAD) study (BP40387), CB2R-TE was measured in blood from healthy participants that received single and multiple 0.75-300 mg doses of RO6868847. Based on clinical RO6868847 pharmacokinetics (PK) and pharmacodynamics (PD; measured by CB2R-TE) data and accounting for the difference between healthy and diabetics subjects, the lower end of the 0.75 mg to 300 mg-dose range is expected to achieve approximately up to half-maximum CB2R-TE, whereas the higher end of that dose range is predicted to achieve maximum CB2R-TE. These doses extend surprisingly higher than the predictions based on the non-clinical models.

In addition to the CB2R-TE whole blood assay, an in vitro LPS whole blood challenge assay was applied using blood from healthy participants that received multiple 300 mg doses of RO6868847 6 in the MAD study. Surprisingly, a signal for attenuation of LPS-induced cytokine release was observed only at the higher end of the dose range, suggesting an additional pharmacological effect that supports the use of doses that provide exposure in the high exposure range.

In summary, the clinical dose range was established based on a combination of the three datasets, the non-clinical, the biomarker readouts for CB2 for target engagement and effects on LPS induced cytokine production in blood from healthy participants, elderly diabetics and age- and demographically matched controls and lastly on the clinical and biomarker data obtained in healthy participants in phase I clinical study.

### BRIEF SUMMARY

A first aspect of the invention relates to the compound (3S)-1-[5-tert-butyl-3-[(1-methyltetrazol-5-yl)methyl]triazolo[4,5-d]pyrimidin-7-yl]pyrrolidin-3-ol for use as agonist of the Cannabinoid Receptor 2, at a dose between 0.75 mg and 300 mg in a patient.

A second aspect of the invention relates to a method for the treatment of an agonist of the Cannabinoid Receptor 2, comprising administering to a patient (in particular a patient in need thereof), (3S)-1-[5-tert-butyl-3-[(1-methyltetrazol-5-yl)methyl]triazolo[4,5-d]pyrimidin-7-yl]pyrrolidin-3-ol at a dose between 0.75 mg and 300 mg.

A third aspect of the invention relates to the use compound (3S)-1-[5-tert-butyl-3-[(1-methyltetrazol-5-yl)methyl]triazolo[4,5-d]pyrimidin-7-yl]pyrrolidin-3-ol in the manufacture of a medicament as agonist of the Cannabinoid Receptor 2 at a dose between 0.75 mg and 300 mg.

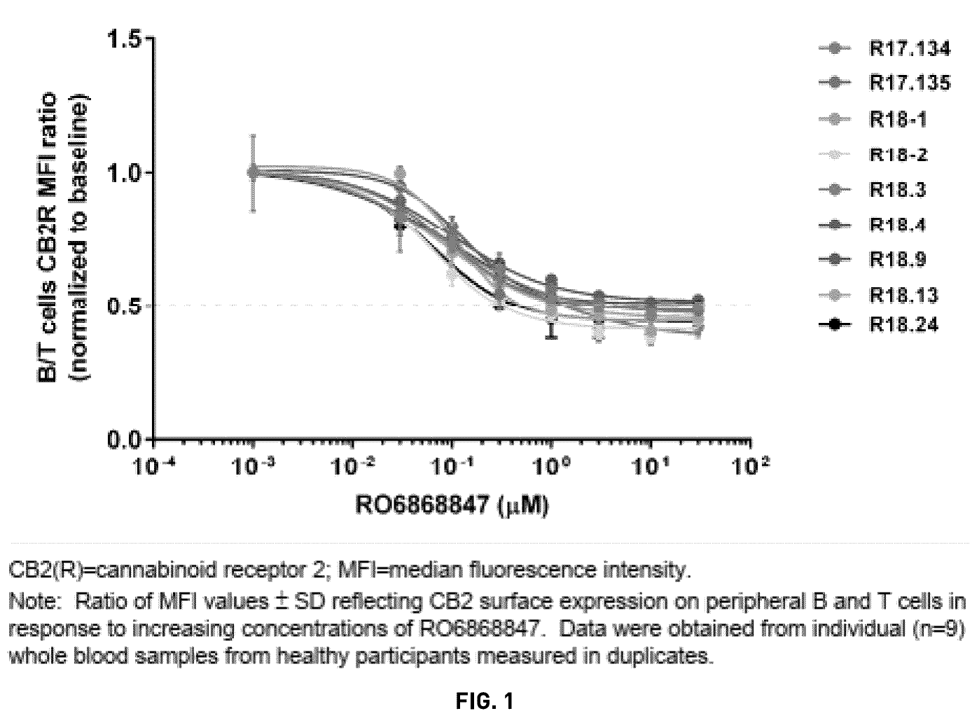

FIG. 1 illustrates Ratio of CB2 Surface Expression on B Cells to T Cells in Whole Blood from Healthy Participants after Exposure to Increasing Concentrations of RO6868847.

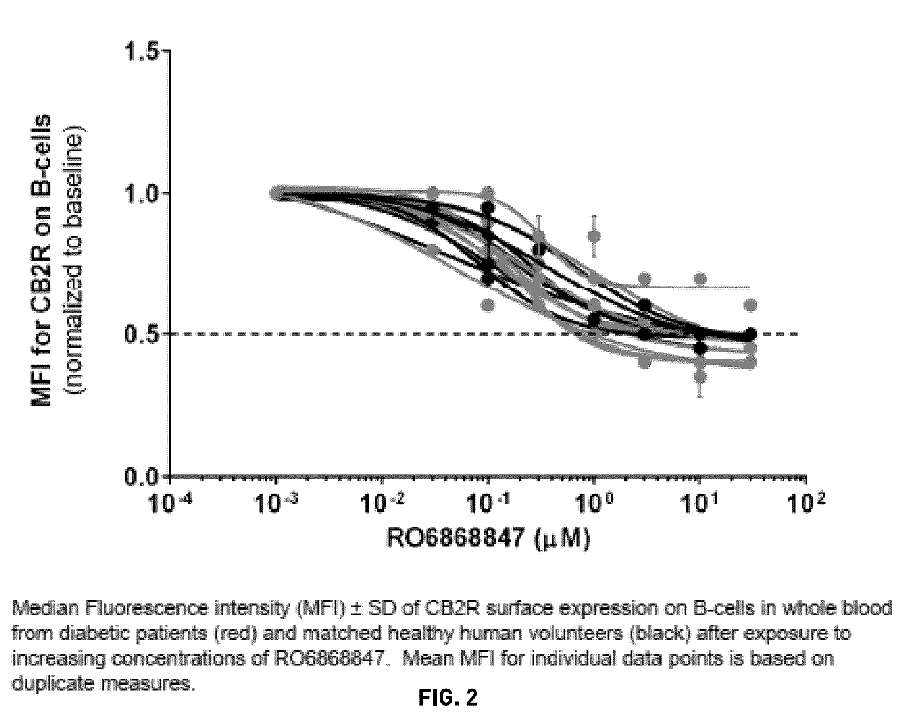

FIG. 2 illustrates IC50 value Determination for RO6868847 in B-cells of Diabetic Patients and Age-Matched Healthy Participants.

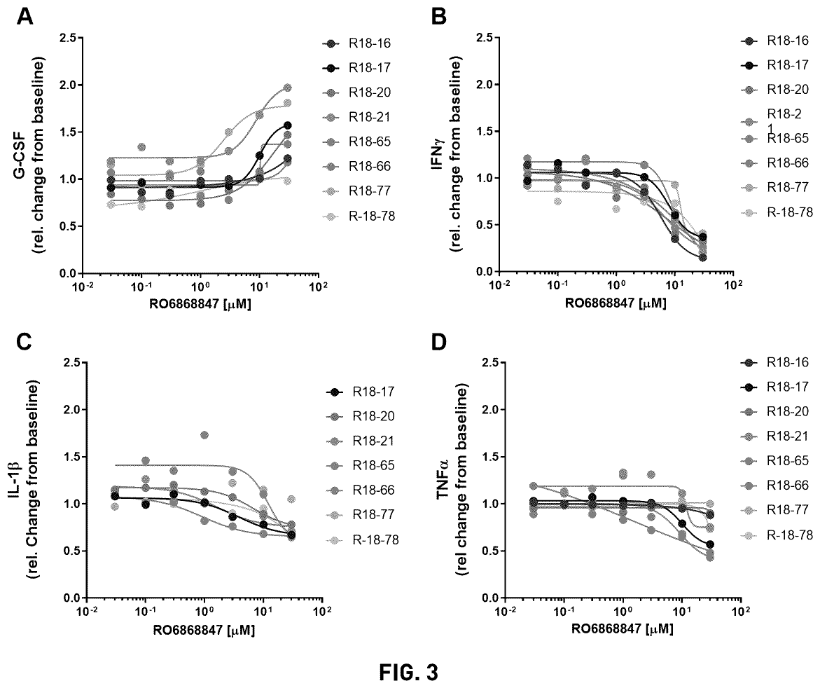

FIG. 3 illustrates IC50 Determination for Effect of RO6868847 on G-CSF, IFNγ, IL1β and TNFα Levels in a LPS Whole Blood Challenge Assay in Healthy Participants In Vitro.

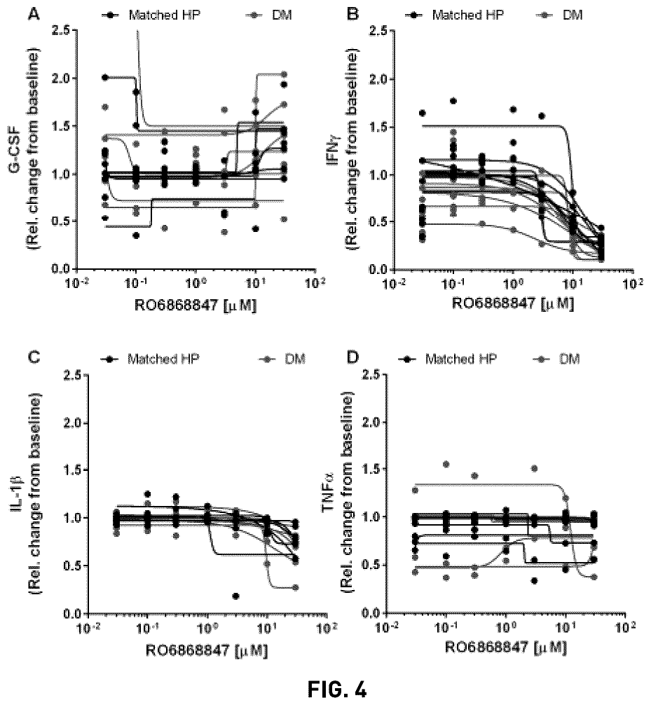

FIG. 4 illustrates IC50 Determination for Effect of RO6868847 on G-CSF, IFNγ, IL1β and TNFα Levels in a LPS Whole Blood Challenge Assay in Diabetic Patients and Matched Healthy Participants In Vitro.

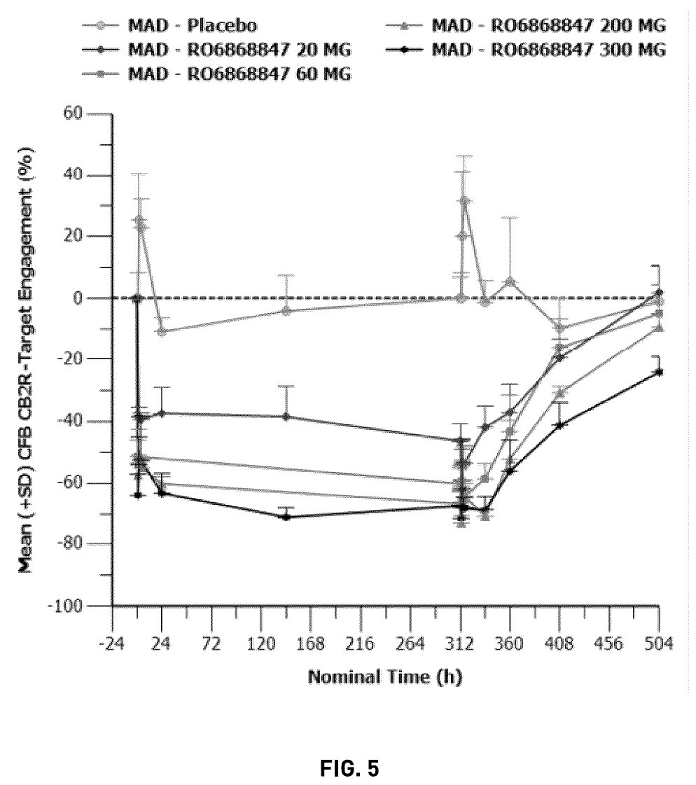

FIG. 5 illustrates Mean (+SD) CB2 Receptor Target Engagement on B-cells (% Change-From-Baseline) Versus Time Profiles After Multiple Oral Dose Administration of 20 mg to 300 mg RO6868847 or Placebo for 14 days.

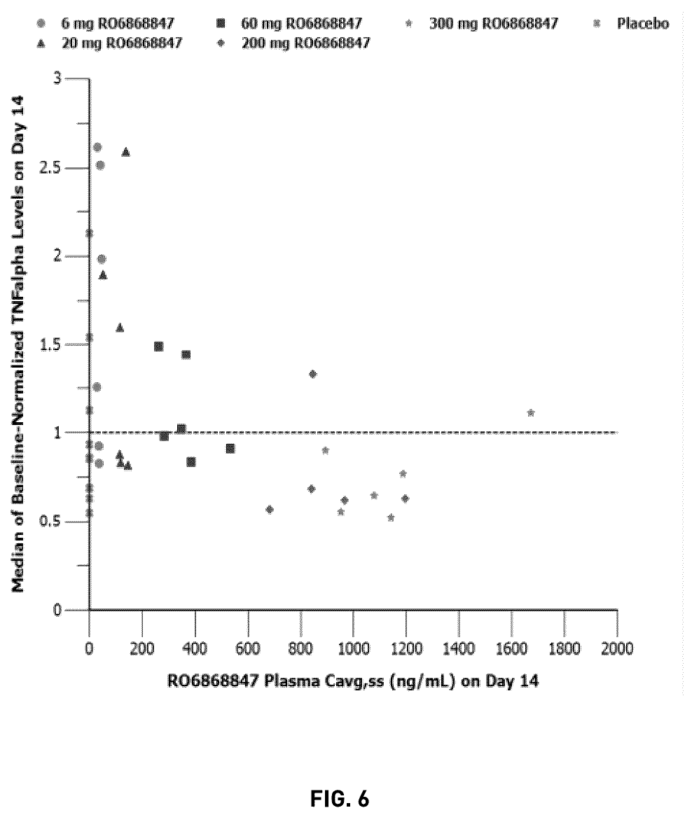

FIG. 6 illustrates Individual Median of Baseline-Normalized TNF-α Levels Versus Plasma Cavg,ss of RO6868847 on Day 14.

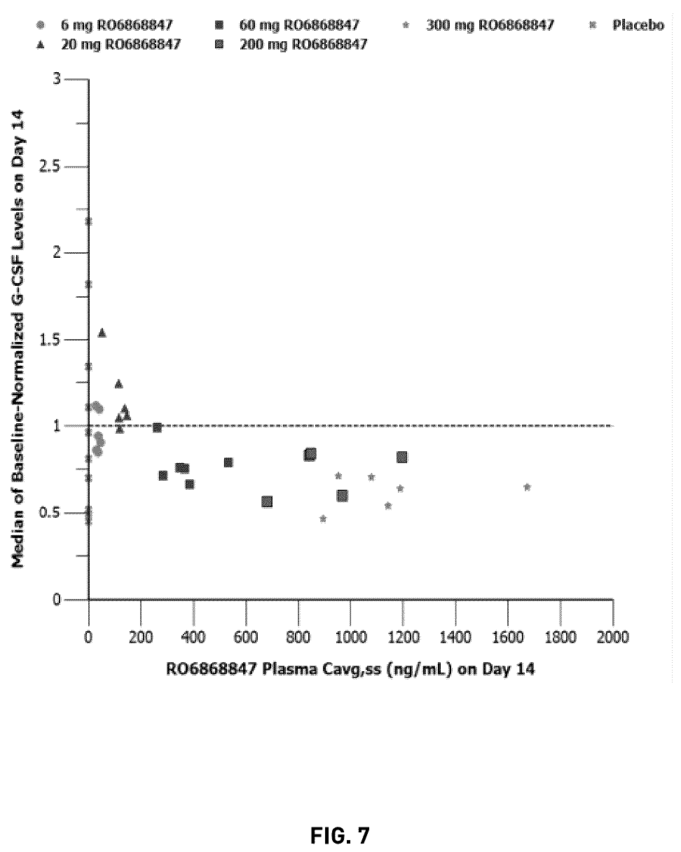

FIG. 7 illustrates Individual Median of Baseline-Normalized G-CSF Levels Versus Plasma Cavg,ss of RO6868847 on Day 14.

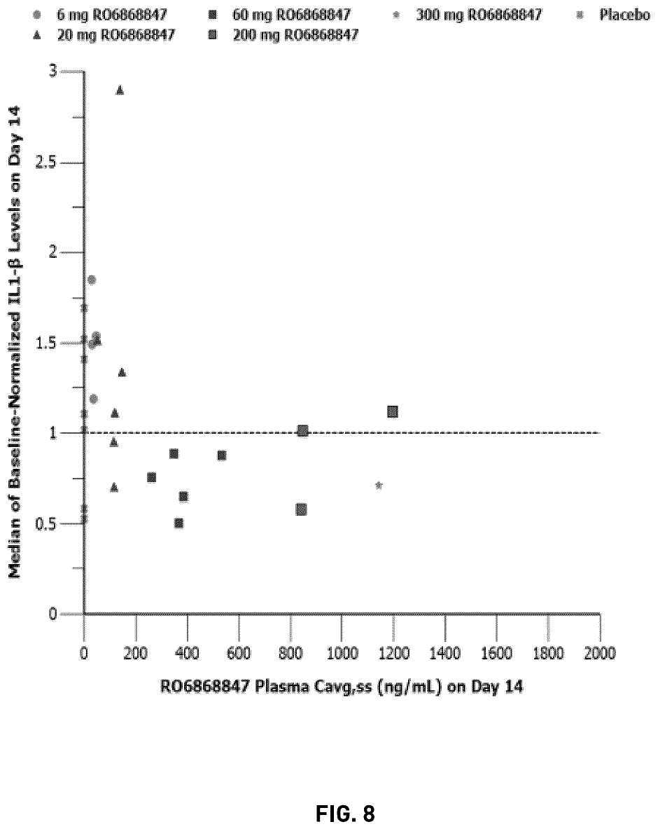

FIG. 8 illustrates Individual Median of Baseline-Normalized IL-1β Levels Versus Plasma Cavg,ss of RO6868847 on Day 14.

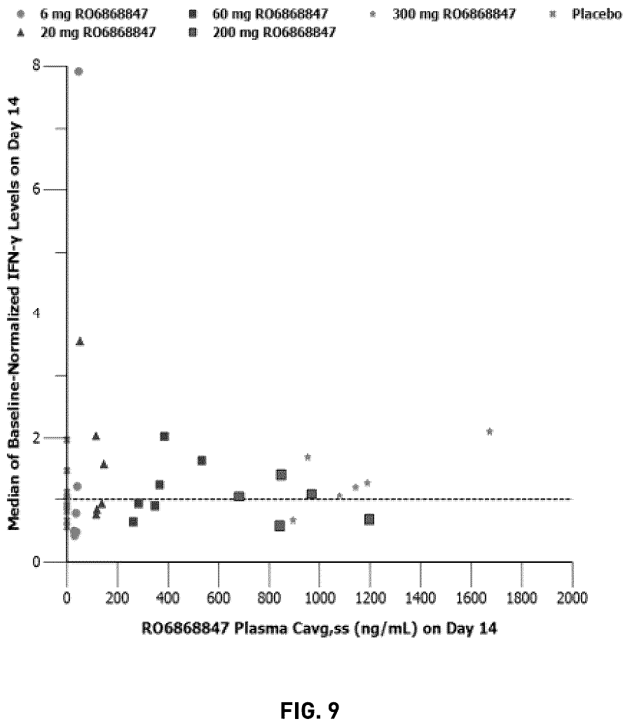

FIG. 9 illustrates Individual Median of Baseline-Normalized IFNγ Levels Versus Plasma Cavg,ss of RO6868847 on Day 14.

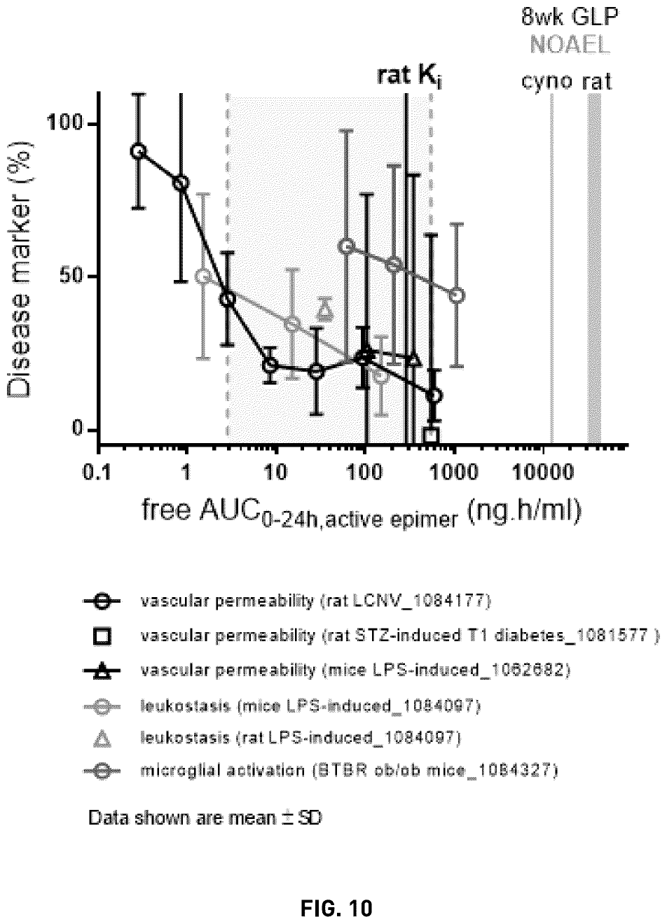

FIG. 10 illustrates Disease Markers versus Free Exposure of the Active S-Epimer RO6868847 in Several Animal Models. AUC₀₋₂₄=area under the concentration-time curve from 0 to 24 hours; cyno=cynomolgus monkey; GLP=Good Laboratory Practice; Kᵢ=equilibrium dissociation constant; LCNV=laser-induced neovascularization; LPS=lipopolysaccharide; NOAEL=no observed adverse effect level; OD=optical density; RO6868847=vicasinabin; STZ=streptozotocin. Disease markers (hyperfluorescence area in mm², OD retina-plasma fluorescein ratio, number of leukocytes per mm² or total length of processes in mm) as a percent of the control value (100% represents the vehicle-treated diabetic control and 0% for vehicle-treated healthy control) in different in vivo PD models as a function of the free AUC₀₋₂₄ of the active S-epimer RO6868847. The rat Kᵢ (33.3 nM=12 ng/mL was extrapolated to a free AUC₀₋₂₄ as follows: free AUC₀₋₂₄=12′24=286 ng·hr/mL. The green area represents the estimated pharmacologically active exposure range in humans.

Unless otherwise stated, the following terms used in the specification and claims have the meanings given below:

“Active pharmaceutical ingredient” or “API” refers to the compound or molecule in a pharmaceutical composition that has a particular biological activity.

“Chronic degenerative disease” refers to a disease in which the function and/or structure of the affected tissues or organs change for the worse over time. Examples of chronic degenerative diseases are, but not limited to, neurodegenerative inflammatory disorders, atherosclerosis, liver fibrosis, or microvascular diabetic complications.

“Diabetes mellitus” or “DM” refers to various forms of glucose metabolic disorders with different etiologies and symptoms. A common characteristic is a relative or absolute insulin deficiency. Diabetes mellitus diseases are characterized by a permanent increase of blood glucose level (hyperglycemia) or by a mistimed utilization of supplied glucose. Diabetes mellitus is subdivided into type I (insulin-dependent; IDDM) and type II (non-insulin-dependent; NIDDM). Diabetes-specific and diabetes-associated chronic complications include microangiopathy, such as retinopathy, nephropathy and neuropathy, polyneuropathy, diabetic foot, disorders of the skeletal, supporting and connective tissue as well as macroangiopathy, especially coronary heart disease, cerebral circulation disorder and peripheral arterial occlusive disease.

“Diabetic retinopathy” or DR refers to a microangiopathy of the eye-ground occurring in diabetes mellitus. Forms of diabetic retinopathy are non-proliferative retinopathy (NPDR) (background retinopathy), such as retinal hemorrhages, microaneurysms, hard exudates, retinal edema with loss of visual acuity, as well as proliferative retinopathy (PDR), in which there is additional occurrence of cotton-wool spots and angiogenesis on and in front of the retina with vitreous hemorrhage due to retinal ischemia from vascular occlusion. Proliferative retinopathy may result in traction retinal detachment, neovascular glaucoma and blindness. With the term DR all stages of PDR and NPDR are encompassed.

“Diabetic macular edema” or DME refers to the swelling of the macula caused by retinal blood vessel leakage that occurs in patients with diabetes. DME is the major cause of vision loss in people with diabetic retinopathy. People with diabetes have a 10 percent risk of developing DME during their lifetime. DME affects up to 30% of people who have had diabetes for more than 20 years. If left untreated, DME can result in moderate to severe vision loss.

“Individual” or “subject” refers to, used interchangeably, a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

“Microvascular diabetic complications” refers to diabetic retinopathy, nephropathy, and neuropathy, which can lead to renal failure, peripheral arterial disease, or limb amputation.

“Non-proliferative diabetic retinopathy” or “NPDR” refers to the early stage of diabetic retinopathy, it is characterized by edema and hard exudates, lipid that has leaked from abnormal blood vessels, in the central retina, resulting in blurred central vision. It may also encompass vascular occlusion, a restriction of blood supply to the retina, as well as an increase in macular edema.

“Patient” refers to a human (such as a male or female human) who is in need of a treatment with RO6868847.

### DETAILED DESCRIPTION

All publications, patent applications, patents and other references mentioned herein are incorporated by reference in their entirety.

The nomenclature used in the present application is based on IUPAC systematic nomenclature, unless indicated otherwise.

Various features and embodiments of the present invention are disclosed herein, however other features of the invention, modifications and equivalents will be apparent to a person skilled in the relevant art, based on the teachings provided. The invention described is not limited to the examples and embodiments provided, various alternatives equivalents will be appreciated by those skilled in the art. As used herein, the singular forms “a”, “an” and “the” include the plural unless the context clearly dictates otherwise. For example, “an” individual will also include “individuals”.

(3S)-1-[5-tert-butyl-3-[(1-methyltetrazol-5-yl)methyl]triazolo[4,5-d]pyrimidin-7-yl]pyrrolidin-3-ol according to the present invention refers to a compound of formula (I)

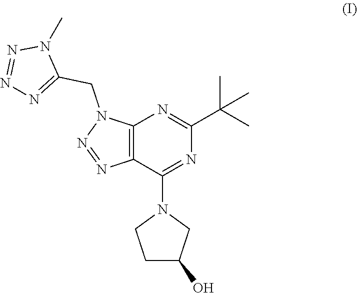 [SMILES: Cn1nnnc1Cn1nnc2c(N3CC[C@H](O)C3)nc(C(C)(C)C)nc21]

also known as RO6868847, Vicasinabin, CAS number 1433361-02-4, methods of making and using the compound are described in WO2022/106669 and WO2012EP71788.

In one embodiment the invention provides (3S)-1-[5-tert-butyl-3-[(1-methyltetrazol-5-yl)methyl]triazolo[4,5-d]pyrimidin-7-yl]pyrrolidin-3-ol for use as agonist of the Cannabinoid Receptor 2, at a dose between 0.75 mg and 300 mg in a patient.

In a particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention of chronic degenerative diseases, at a dose between 0.75 mg and 300 mg in a patient.

In a particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention of neurodegenerative inflammatory disorders, atherosclerosis, liver fibrosis, or microvascular diabetic complications, at a dose between 0.75 mg and 300 mg in a patient.

In a particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention of microvascular diabetic complications, wherein microvascular diabetic complications are diabetic retinopathy, diabetic macular edema, or non-proliferative diabetic retinopathy at a dose between 0.75 mg and 300 mg in a patient.

In a particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention of non-proliferative diabetic retinopathy (NPDR), at a dose between 0.75 mg and 300 mg in a patient.

In a particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention of proliferative diabetic retinopathy (PDR), at a dose between 0.75 mg and 300 mg in a patient.

In a particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention of diabetic macular edema (DME), at a dose between 0.75 mg and 300 mg in a patient.

In a particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention of diabetic nephropathy (DN), at a dose between 0.75 mg and 300 mg in a patient.

In a more particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention as described hereinabove, wherein the patient is a human (such as a male or female human).

In a more particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention as described hereinabove, wherein it is orally administered.

In a more particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention as described hereinabove, wherein the patient is a human (such as a male or female human), wherein the compound is being administered, in particular administered orally, at a dose between 0.75 mg and 300 mg.

In a more particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention as described hereinabove, wherein it is administered once a day.

In a more particular embodiment, the invention provides (3S)-1-[5-tert-butyl-3-[(1-methyltetrazol-5-yl)methyl]triazolo[4,5-d]pyrimidin-7-yl]pyrrolidin-3-ol for use in the treatment or prevention as described herein above, wherein it is administered in combination with other treatments, in particular in combination with laser, anti-VEGF treatments, or other treatments for diabetic retinopathy.

In one embodiment, the invention provides a method for the treatment of an agonist of the Cannabinoid Receptor 2, comprising administering to a patient (in particular a patient in need thereof), (3S)-1-[5-tert-butyl-3-[(1-methyltetrazol-5-yl)methyl]triazolo[4,5-d]pyrimidin-7-yl]pyrrolidin-3-ol at a dose between 0.75 mg and 300 mg.

In a particular embodiment, the invention provides a method for the treatment or prevention of chronic degenerative diseases, comprising administering to a patient (in particular a patient in need thereof), (3S)-1-[5-tert-butyl-3-[(1-methyltetrazol-5-yl)methyl]triazolo[4,5-d]pyrimidin-7-yl]pyrrolidin-3-ol at a dose between 0.75 mg and 300 mg.

In a particular embodiment, the invention provides a method for the treatment or prevention of neurodegenerative inflammatory disorders, atherosclerosis, liver fibrosis, or microvascular diabetic complications, comprising administering to a patient (in particular a patient in need thereof), (3S)-1-[5-tert-butyl-3-[(1-methyltetrazol-5-yl)methyl]triazolo[4,5-d]pyrimidin-7-yl]pyrrolidin-3-ol at a dose between 0.75 mg and 300 mg.

In a particular embodiment, the invention provides a method for the treatment or prevention of microvascular diabetic complications, wherein microvascular diabetic complications are diabetic retinopathy, diabetic macular edema, or non-proliferative diabetic retinopathy, comprising administering to a patient (in particular a patient in need thereof), (3S)-1-[5-tert-butyl-3-[(1-methyltetrazol-5-yl)methyl]triazolo[4,5-d]pyrimidin-7-yl]pyrrolidin-3-ol at a dose between 0.75 mg and 300 mg.

In a particular embodiment, the invention provides a method for the treatment or prevention of non-proliferative diabetic retinopathy (NPDR), comprising administering to a patient (in particular a patient in need thereof), (3S)-1-[5-tert-butyl-3-[(1-methyltetrazol-5-yl)methyl]triazolo[4,5-d]pyrimidin-7-yl]pyrrolidin-3-ol at a dose between 0.75 mg and 300 mg.

In a particular embodiment, the invention provides a method for the treatment or prevention of proliferative diabetic retinopathy (PDR), comprising administering to a patient (in particular a patient in need thereof), (3S)-1-[5-tert-butyl-3-[(1-methyltetrazol-5-yl)methyl]triazolo[4,5-d]pyrimidin-7-yl]pyrrolidin-3-ol at a dose between 0.75 mg and 300 mg.

In a particular embodiment, the invention provides a method for the treatment or prevention of diabetic macular edema (DME), comprising administering to a patient (in particular a patient in need thereof), (3S)-1-[5-tert-butyl-3-[(1-methyltetrazol-5-yl)methyl]triazolo[4,5-d]pyrimidin-7-yl]pyrrolidin-3-ol at a dose between 0.75 mg and 300 mg.

In a particular embodiment, the invention provides a method for the treatment or prevention of diabetic nephropathy (DN), comprising administering to a patient (in particular a patient in need thereof), (3S)-1-[5-tert-butyl-3-[(1-methyltetrazol-5-yl)methyl]triazolo[4,5-d]pyrimidin-7-yl]pyrrolidin-3-ol at a dose between 0.75 mg and 300 mg.

In a more particular embodiment, the invention provides a method for the treatment or prevention as described hereinabove, particularly wherein the patient is a human (such as a male or female human).

In a more particular embodiment, the invention provides a method for the treatment or prevention as described hereinabove, wherein it is orally administered.

In a more particular embodiment, the invention provides a method for the treatment or prevention as described hereinabove, wherein the patient is a human (such as a male or female human), wherein the compound is being administered, in particular administered orally, at a dose between 0.75 mg and 300 mg.

In a more particular embodiment, the invention provides a method for the treatment or prevention as described hereinabove, wherein it is administered once a day.

In a more particular embodiment, the invention provides a method for the treatment or prevention as described hereinabove, wherein it is administered in combination with other treatments, in particular in combination with laser, anti-VEGF treatments, or other treatments for diabetic retinopathy.

In one embodiment, the invention provides the use of compound (3S)-1-[5-tert-butyl-3-[(1-methyltetrazol-5-yl)methyl]triazolo[4,5-d]pyrimidin-7-yl]pyrrolidin-3-ol in the manufacturing of a medicament as agonist of the Cannabinoid Receptor 2 at a dose between 0.75 mg and 300 mg.

In a particular embodiment, the invention provides the use compound (3S)-1-[5-tert-butyl-3-[(1-methyltetrazol-5-yl)methyl]triazolo[4,5-d]pyrimidin-7-yl]pyrrolidin-3-ol for the manufacturing of a medicament for the treatment or prevention of chronic degenerative diseases at a dose between 0.75 mg and 300 mg.

In a particular embodiment, the invention provides the use compound (3S)-1-[5-tert-butyl-3-[(1-methyltetrazol-5-yl)methyl]triazolo[4,5-d]pyrimidin-7-yl]pyrrolidin-3-ol for the manufacture of a medicament for the treatment or prevention of neurodegenerative inflammatory disorders, atherosclerosis, liver fibrosis, or microvascular diabetic complications at dose between 0.75 mg and 300 mg.

In a particular embodiment, the invention provides the use compound (3S)-1-[5-tert-butyl-3-[(1-methyltetrazol-5-yl)methyl]triazolo[4,5-d]pyrimidin-7-yl]pyrrolidin-3-ol for the manufacture of a medicament for the treatment or prevention of microvascular diabetic complications, wherein microvascular diabetic complications are diabetic retinopathy, diabetic macular edema, or non-proliferative diabetic retinopathy at dose between 0.75 mg and 300 mg.

In a particular embodiment, the invention provides the use compound (3S)-1-[5-tert-butyl-3-[(1-methyltetrazol-5-yl)methyl]triazolo[4,5-d]pyrimidin-7-yl]pyrrolidin-3-ol for the manufacture of a medicament for the treatment or prevention of non-proliferative diabetic retinopathy (NPDR) at dose between 0.75 mg and 300 mg.

In a particular embodiment, the invention provides the use compound (3S)-1-[5-tert-butyl-3-[(1-methyltetrazol-5-yl)methyl]triazolo[4,5-d]pyrimidin-7-yl]pyrrolidin-3-ol for the manufacture of a medicament for the treatment or prevention of proliferative diabetic retinopathy (PDR) at dose between 0.75 mg and 300 mg.

In a particular embodiment, the invention provides the use compound (3S)-1-[5-tert-butyl-3-[(1-methyltetrazol-5-yl)methyl]triazolo[4,5-d]pyrimidin-7-yl]pyrrolidin-3-ol for the manufacture of a medicament for the treatment or prevention of diabetic macular edema (DME) at dose between 0.75 mg and 300 mg.

In a particular embodiment, the invention provides the use compound (3S)-1-[5-tert-butyl-3-[(1-methyltetrazol-5-yl)methyl]triazolo[4,5-d]pyrimidin-7-yl]pyrrolidin-3-ol for the manufacture of a medicament for the treatment or prevention of diabetic nephropathy (DN) at dose between 0.75 mg and 300 mg.

In a more particular embodiment, the invention provides the use of compound (3S)-1-[5-tert-butyl-3-[(1-methyltetrazol-5-yl)methyl]triazolo[4,5-d]pyrimidin-7-yl]pyrrolidin-3-ol as described hereabove, particularly wherein the patient is a human (such as a male or female human).

In a more particular embodiment, the invention provides the use of compound (3S)-1-[5-tert-butyl-3-[(1-methyltetrazol-5-yl)methyl]triazolo[4,5-d]pyrimidin-7-yl]pyrrolidin-3-ol as described hereabove, wherein it is orally administered.

In a more particular embodiment, the invention provides the use of compound (3S)-1-[5-tert-butyl-3-[(1-methyltetrazol-5-yl)methyl]triazolo[4,5-d]pyrimidin-7-yl]pyrrolidin-3-ol as described hereabove, wherein the patient is a human (such as a male or female human), wherein the compound is being administered, in particular administered orally, at a dose between 0.75 mg and 300 mg.

In a more particular embodiment, the invention provides the use of compound (3S)-1-[5-tert-butyl-3-[(1-methyltetrazol-5-yl)methyl]triazolo[4,5-d]pyrimidin-7-yl]pyrrolidin-3-ol as described hereabove, wherein it is administered once a day.

In a more particular embodiment, the invention provides the use of compound (3S)-1-[5-tert-butyl-3-[(1-methyltetrazol-5-yl)methyl]triazolo[4,5-d]pyrimidin-7-yl]pyrrolidin-3-ol as described hereabove, wherein it is administered in combination with other treatments, in particular in combination with laser, anti-VEGF treatments, or other treatments for diabetic retinopathy.

### EXAMPLES

### Nonclinical Studies

### CB2 In Vitro Pharmacology

Pharmacological studies were performed in vitro and in vivo to investigate the potency, selectivity and activity of RO6868847. Pharmacological effects were determined in vivo in two different diabetic animal models which include the streptozotocin (STZ)-induced rat Type 1 diabetes and genetic Type 2 diabetes mouse models as well as in two acute models with acute inflammatory stimulus (LPS-injections) or laser injury (CNV). Additional in vitro pharmacology studies were performed to explore the pharmacological effect of RO6868847 and the suitability of the assays as biomarkers of target engagement and pharmacodynamic effect (PD) in human whole blood obtained from either healthy volunteers or diabetic patients.

RO6868847 binds to human CB2 with a Ki of 51.3 nM and 62.8 nM, when measured with Chinese hamster ovary (CHO) cells expressing recombinant CB2 or with U968M cells expressing endogenous CB2. Among several species tested, RO6868847 displays highest affinity to human and cynomolgus monkey CB2, with comparable Ki values of 51.3 nM and 66.8 nM, respectively. Table 3 summarizes the affinity of RO6868847 to CB2 receptors. On the basis of physiological expression levels and receptor activity states in endogenous systems, the human Ki of 62.8 nM in U698M cells is proposed to be used for human dose predictions.

TABLE 3
Binding potencies of RO6868847 to CB2

| Species | Cells | IC₅₀ ± SE (nM) ^{a} | Kᵢ ± SE (nM) ^{b} |
|---|---|---|---|
| Human | CHO cells | 117.5 ± 26.3 | 51.3 ± 16.2 |
| Cynomolgus | CHO cells | 102.7 ± 17.2 | 66.8 ± 15.9 |
| monkey | | | |
| Human | U698M | 92.2 ± 13.1 | 62.8 ± 12.5 |

CB2 = cannabinoid receptor 2; CHO = Chinese hamster ovary; K = equilibrium dissociation constant.
^{a} IC₅₀ refers to the molar concentration of an inhibitor that displaces 50% of the radioligand. IC₅₀ values reported are means ± SE of three independent experiments each performed in duplicates.
^{b} K refers to the equilibrium dissociation constant for the unlabeled ligand.

### Selectivity for CB2 Over CB1 and Other Targets

Inhibition of cAMP accumulation in Chinese hamster ovary cells stably expressing recombinant CB2 was used to determine the functional potency of RO6868847 for different CB2 species and the selectivity over the closely related CB1. RO6868847 is a potent, full agonist for human CB2 and displays similar potency for CB2 in other species including the cynomolgus monkey, rat, and mouse. Table 4 summarizes the potency from different CB2 species.

TABLE 4
Agonist potencies of RO6868847 for Forskolin-Stimulated
cAMP inhibition in stably transfected CHO cells

| Species | Cells | IC₅₀ ± SE (nM) ^{a} | Eₘₐₓ ^{b} |
|---|---|---|---|
| Human | CHO cells | 2.81 ± 0.28 | 103.1% |
| Cynomolgus | CHO cells | 1.06 ± 0.08 | 94.6% |
| monkey | | | |
| Rats | CHO cells | 0.58 ± 0.04 | 94.7% |
| Human | CHO cells | 2.60 ± 0.14 | 101.1% |

cAMP = cyclic adenosine monophosphate; CB2 = cannabinoid receptor 2; CHO = Chinese hamster ovary; Eₘₐₓ = maximum effect.
^{a} EC₅₀ refers to the molar concentration of an agonist activating 50% of the maximal effect. EC₅₀ values reported are means ± SE of four independent experiments each performed in duplicates.
^{b} Maximal activity Eₘₐₓ of RO6868847 is normalized to the effect produced by 10 μM CP55′940.

RO6868847 revealed no affinity for human CB1, and no receptor activation or inhibition was seen at the highest concentration of 10 μM tested, resulting in a binding selectivity of >195-fold and functional selectivity of >3600-fold over human CB1 (see Table 5).

TABLE 5
CB2 selectivity of RO6868847 over human CB1

| | | | | CB2 | CB1/ |
|---|---|---|---|---|---|
| | | EC₅₀ | IC₅₀ | Reference | CB2 |
| Assay | Kᵢ (nM) ^{a} | (nM) ^{b} | (nM) ^{c} | Value (nM) | Ratio |
| Human CB1 | >10,000 | — | — | K = S1.3 | >195 |
| binding assay | (n = 3) | | | (Table 3) | |
| Human CB1 | — | >10,000 | — | EC₅₀ = 2.81 | >3600 |
| cAMP assay | | (n = 3) | | (Table 4) | |
| Human CB1 | — | — | >10,000 | — | — |
| cAMP assay | | | (n = 1) | | |

cAMP = cyclic adenosine monophosphate; CB1 = cannabinoid receptor 1; CB2 = cannabinoid receptor 2; K = equilibrium dissociation constant.
^{a} Kᵢ refers to the equilibrium dissociation constant for the unlabeled ligand.
^{b} EC₅₀ refers to the molar concentration of an agonist activating 50% of the maximal effect.
^{c} IC₅₀ refers to the molar concentration of an agonist inhibiting 50% of the maximal effect induced by 10 μM CP55′940.
Note:
n indicates the number of independent experiments each performed in triplicates.

The in vitro pharmacology profile of RO6868847 was evaluated at concentration of 30 μM on panels including 131 targets along with 57 CNS- and abuse potential-specific targets.

RO6868847 showed a high selectivity for CB2 in all the panels. Further in vitro assays assessed the selectivity of RO6868847 for 78 additional receptors and ion channels using radioligand binding assays in which RO6868847 demonstrated excellent selectivity for CB2. In functional cellular assays, no activity of RO6868847 was detected at both human and mouse 15-lipoxygenase, MAGL, diacylglycerol lipase, or FAAH enzymes of the endocannabinoid pathway (10 μM in mouse and up to 500 μM in human targets). These results suggest a low liability of RO6868847 for off-target effects, and as such, no follow-up investigations in functional assays were considered necessary.

### Example 1: In Vitro Target Engagement by Monitoring CB2 Surface Expression on Peripheral B Cells in Whole Blood from Healthy Participants and Diabetic Patients

To monitor target engagement by RO6868847 in humans, a flow cytometry assay was developed utilizing an in-house monoclonal anti-human CB2 antibody in combination with established blood cell markers (CD45, CD14, CD3, and CD19) (1078220). Assessment of surface expression of CB2 on peripheral leukocyte subsets in whole blood from healthy participants utilizing this assay confirmed published findings by Castaneda and colleagues (Castaneda et al. 2013) that B cells show a low but robust surface expression of CB2, while surface expression of CB2 was not detected in other peripheral blood cell populations such as T cells, granulocytes, or monocytes. Therefore, B cells were chosen to monitor CB2 surface expression in order to evaluate any possible drug effect on this measure in peripheral blood. As with most GPCR agonists, RO6868847 is expected to trigger CB2 receptor internalization upon CB2 activation, resulting in decreased surface detection. In order to establish this measure as a surrogate target engagement biomarker, freshly isolated whole blood samples (n=9) from healthy participants were incubated in duplicates in vitro for 1 hour at 37° C. with eight different increasing concentrations of RO6868847 and were subsequently subjected to the flow cytometry assay (1078220). The data for the individual donors (n=9) is expressed as a ratio of CB2 surface expression on B-cells to T cells and normalized relative to the same ratio in whole blood of the respective donor without drug incubation (see FIG. 1), revealing a concentration-dependent reduction of CB2 surface expression on B-cells upon RO6868847 treatment.

Utilizing the data from the 9 individual donors a mean IC50 (TE)±SD=96.5±27.1 nM was estimated together with a respective mean Emax=55.0±4.6% (34.6 ng/mL). Furthermore, this in vitro data suggested that monitoring target engagement in human plasma using this flow cytometry assay may be feasible in human peripheral blood and was therefore used in the Phase I Study BP40387 in healthy participants.

The availability of this in vitro target engagement assay also enabled us to explore the target engagement—exposure response in blood samples obtained from patients with diabetic mellitus (Type 1 and Type 2) and healthy controls matched for age, gender and BMI (Table 6) (1097891). This population was selected as it is similar to the envisaged target patient population for the proof-of-concept Phase II trial, encompassing diabetic patients according to definition by the World Health Organization and/or the American Diabetes Association.

TABLE 6
Baseline clinical characteristics of diabetic and
matched healthy participants

| Characteristic | Diabetics | Matched Healthy* | p-value |
|---|---|---|---|
| Subjects | 9 | 8 | nd |
| Female (%) | 1 (11.1%) | 1 (12.5%) | |
| Male (%) | 8 (88.9%) | 7 (87.5%) | |
| Mean Age ± SD | 56 ± 13 | 52 ± 11 | 0.58 |
| in years | | | |
| Weight (kg) | 94 (81-95) | 98 (83.6-102.8) | 0.49 |
| BMI (kg/m²) | 26.4 (25.4-32.0) | 29 (26.3-31.7) | 0.90 |
| CRP (mg/l) | 0.9 (0.4-2.3) | 1.3 (1.1-1.6) | 0.33 |
| Fasting glucose | 6.3 (6.0-6.5) | 4.5 (4.2-4.9) | 0.02 |
| (mmol/l) | 7.0 (7.1.7.2) | 5.3 (5.1-5.4) | <0.0001 |
| HbA1c level | | | |
| (mmol/l) | | | |

BMI = body mass index; CRP = C reactive protein; HbA1c = hemoglobin A1c; SD = standard deviation; nd = not determined.
If not stated otherwise, median values and interquartile range (in brackets) are given. Level of significance was calculated using Mann-Whitney U-Test
*Matched healthy controls with respect to age, gender and BMI.

These experiments showed that also in whole blood obtained from diabetic patients and matched healthy participants, RO6868847 induces a dose-dependent CB2 receptor internalization on the surface of B-cells compared to the CB2 receptor level without exposure to RO6868847 (FIG. 2).

However, by comparing the respective mean IC50±SD values for CB2 receptor level for the three groups of participants (Table 7) the data show a trend that diabetic (DM) patients and matched healthy participants may require about 2-3 fold higher exposure to achieve half maximum internalization of the CB2 receptor on B-cells (One-way Anova with Tukeys multiple comparsion correction, p=0.0575) compared to previously analyzed healthy participants. This was considered during dose-selection for the Phase 2 Study.

TABLE 7
Comparison of IC50 (CB2 receptor on B-cells) in Diabetic (DM) and
matched healthy participants with previous data (FIG. 1)

| | Mean IC₅₀ | Mean | |
|---|---|---|---|
| Group/ | (CB2 on B-cells) ± | Eₘₐₓ ± SD | Fold change |
| Diagnosis | SD (nM) | (%) | vs HPs |
| Healthy | 96.5 ± 27.1 (n = 9) | 55.0 ± 4.6 (n = 9) | 1 |
| Participants | | | |
| Matched | 253.3 ± 206.2 (n = 8) | 55.7 ± 6.8 (n = 8) | 2.6 x |
| Healthy | | | |
| DM | 172.5 ± 82.0 (n = 8) | 56.2 ± 11.7 (n = 8) | 1.8x |

Note:
One diabetic patient was exluded from the calculation of the averages IC₅₀ and Eₘₐₓ since the individual IC50 was an outlier (~10 higher).

### Example 2: In Vitro Pharmacodynamics Effect of RO6868847 in an LPS Challenge Assay in Whole Blood from Healthy Volunteers and Diabetic Patients

In order to explore any putative anti-inflammatory pharmacodynamic effect mediated by the CB2 agonist RO6868847 in a potentially clinically relevant human sample, an ex vivo whole blood lipopolysacharid (LPS) challenge assay was performed. Sodium heparin whole blood samples from healthy participants (n=8) were incubated in vitro for 1 hour at 37° C. with eight increasing concentrations of RO6868847 (0-30 μM) and exposed to LPS (100 ng/ml). After 18 h at 37° C. supernatants were analyzed for alterations in a range of chemokines and cytokines using Luminex-bead based multiplex ELISAs. These experiments revealed a dose-dependent alteration of specific chemokines and cytokines levels (decrease of IFNγ, IL1β, TNFα and elevation of G-CSF) post LPS stimulation in whole blood and thus suggest—at high exposures—an overall anti-inflammatory/immune modulatory downstream pharmacodynamics effect of the CB2 agonist in vitro.

In addition, this in vitro LPS whole blood challenge assay was also used in samples from diabetic (Type I and Type II) and matched healthy participants (table 6). In order to explore whether the exposure (0-30 μM)-response relationship of RO6868847 and the respective alterations in cytokine levels may be changed by any chronic inflammatory conditions in diabetes (DM) and/or in elderly healthy participants (HP), who are anticipated to be the study population of the Phase II proof of concept Study.

In diabetic patients and matched healthy participants a dose-dependent attenuation of IFNγ by RO6868847 and a response of IL1β at high exposures in a few participants was observed (FIG. 4) confirming the previous in vitro observations in healthy participants (FIG. 3). However, for G-CSF and TNFα levels an overall drug response in the explored exposure range could not be concluded due to the observed high variability and the fact that only for one or two individual samples EC50/IC50 values could be calculated (FIG. 4, Table 8).

TABLE 8

| | EC₅₀ | IC₅₀ | IC₅₀ | IC₅₀ |
|---|---|---|---|---|
| Group/ | (G-CSF)/μM | (IFNγ)/μM | (IL-1β)/μM | (TNFα)/μM |
| Diagnosis | (n) | (n) | (n) | (n) |
| HV | 7.97 ± 6.71 | 10.9 ± 8.9 | 5.02 ± 4.58 | 7.00 ± 4.61 |
| | (5/8) | (6/8) | (5/8) | (3/8) |
| Matched | n. calc. | 12.41 ± 4.19 | n. calc. | n. calc. |
| HVs | (1/8) | (4/8) | (0/8) | (0/8) |
| DM | n. calc. | 6.91 ± 2.29 | 29.4 ± 24.1 | n. calc. |
| | (2/9) | (7/9) | (3/9) | (1/9)) |

Numbers in brackets indicate the number of subjects vs the total per group for which an EC₅₀ or IC₅₀ curve could be fitted. n.calc. = not calculated. Mean and SD were only calculated, if n ≥ 3.

Overall, this data support a general immune modulatory/anti-inflammatory effect of RO6868847 also in elderly and diabetics. It remains to be explored whether these effects are similar but of different magnitude, due to a less sensitive immune response, or whether the differences are more significant.

### Exposure-Response Analysis Based on In Vivo Pharmacodynamics

Several animal models demonstrated effects of Vicasinabin related to the DR pathophysiology, including inhibition of leukostasis in the eye (rodent diabetic and non-diabetic model), reduction of retinal hyperfluorescence (rodent laser injury model), and preservation of retinal permeability, function, and microglial morphology (rodent diabetic models). The diversity of the tested models (including acute challenge versus disease-modifying models) and the absence of a recommended nonclinical DME/DR model with proven clinical translatability presents a challenge for the estimation of pharmacologically active exposure in humans.

FIG. 10 shows the disease markers as a percent of the control value in different in vivo PD models as a function of the free AUC₀₋₂₄ of the active S-epimer Vicasinabin. Exposure was derived from satellite animals or extrapolated from previous PK or PD studies in the same species.

Based on this integrated assessment, the pharmacologically active exposure range observed in rats spans 2 logs, with an upper bound (unbound AUC₀₋₂₄ [S-epimer]=546 ng·hr/mL) corresponding to the full effect at 10 mg/kg in a rat STZ-induced Type 1 diabetes model and the lower bound (unbound AUC₀₋₂₄ [S-epimer]=2.8 ng·hr/mL) corresponding to 50% decrease in disease marker (hypoerfluorescence area) in a laser-induced NV rat model. The unbound AUC₀₋₂₄ leading to plasma concentrations close to the in vitro rat Kᵢ represents 52% of the upper bound of the observed pharmacologically active exposure in rats.

### Effects in Humans

Completed and ongoing Phase I/II clinical studies of RO6868847 are summarized below. All studies in the clinical development program are detailed in Appendix 4 and have been conducted in accordance with the principles of Good Clinical Practice (GCP). Study BP40387 was a Phase I study that was conducted in healthy male and female participants, and included 5 parts. Part 1 (SAD) consisted of an adaptive, single-ascending-dose, investigator/participant-blind, randomized, placebo-controlled, parallel study to investigate the safety, tolerability, and pharmacokinetics of RO6868847. Part 2 (FE), was designed as an open-label, randomized, two-period crossover, single dose investigation of the effect of food on the pharmacokinetics of RO6868847. Part 3 (MAD), was an adaptive, multiple-ascending-dose, investigator/participant-blind, randomized, placebo-controlled, parallel study to investigate the safety, tolerability, and pharmacokinetics of RO6868847 administered once-daily for 14 days. Part 4 (DDI-CYP Induction), was a non-randomized, open-label, fixed-sequence, two-period study to explore RO6868847-mediated CYP induction at 300 mg administered once-daily for 14 days, using midazolam, repaglinide, and bupropion as probe substrates for CYP3A, CYP2C8, and CYP2B6, respectively. Part 5 (DDI—Transporter-mediated Inhibition), was a non-randomized, open-label, fixed-sequence, four-period study to investigate inhibitory effects of RO6868847 on drug transporters, using metformin and atorvastatin as probe substrates for OCT2 and MATE2-K, and OATP1B1, respectively. Overall, in Part 1 (SAD), a total of 33 healthy participants received single oral doses of RO6868847 ranging between 0.75 mg and 300 mg, and 12 participants received placebo under fasting conditions. In Part 2 (FE), 8 participants received a single oral dose of 100 mg RO6868847 once after an overnight fast of at least 10 hours and once within 30 minutes after starting a high-calorie, high-fat breakfast. In Part 3 (MAD), a total of 30 participants received multiple oral doses of 6 mg to 300 mg RO6868847 QD for 14 days, and 10 participants received placebo.

### Clinical Pharmacokinetics

In Study BP40387, RO6868847 was rapidly absorbed with median maximum plasma concentration (Tmax) between 1 to 4 hours under fasted conditions. No pronounced deviations from dose-proportionality were observed for Cmax and AUCInf/tau up to 100 mg. However, for doses beyond 100 mg Cmax and AUCInf/tau appeared to increase in a slightly less than dose-proportional fashion. AUCtau at steady-state drug was in good agreement with AUCInf after single dose administration, indicating absence of relevant PK time dependencies. Following multiple dose administration of RO6868847 once-daily, steady-state was on average achieved approximately after the second day of dosing with only minor accumulation across the dose range investigated. Renal excretion of RO6868847 following single and multiple dose administration was minor, accounting on average for less than 5% of the dose administered. Overall, food had no relevant effect on the pharmacokinetics of RO6868847.

In Part 3 (MAD), co-administration of a single oral dose of 0.100 mg midazolam with multiple oral doses of 6 mg to 300 mg RO6868847 once daily for 14 days, showed that RO6868847 is a weak inducer of CYP3A at dose of 300 mg administered, but not at the lower doses tested. In Study BP40387, single and multiple oral dose administration of RO6868847 lead to a reversible, dose-dependent and sustained CB2 receptor target engagement on B-cells in blood.

Pharmacokinetics after Single- and Multiple-Dose Oral Administration to Healthy Subjects Single Dose Plasma Pharmacokinetics

In Part 1 (SAD) of Study BP40387, in the fasted state, RO6868847 was overall rapidly absorbed; however, the Tmax appeared to shift with increasing dose. Individual Tmax ranged between 1 to 6 hours at 100 mg and 300 mg RO6868847. The main plasma PK parameters of RO6868847 are summarized in Table 20. AUCinf and Cmax increased up to 100 mg of RO6868847 approximately dose-proportionally; however, above 100 mg to 300 mg RO6868847, Cmax and AUCinf increased in a slightly less than dose-proportional manner. After reaching peak plasma concentrations (Cmax), RO6868847 plasma concentrations declined in a mono-exponential fashion, with an apparent terminal elimination half-life of RO6868847 ranging from 6.95 to 18.5 hours. In addition, with increasing dose variability in half-life increased, due to individual participants exhibiting a bi-phasic decline after peak plasma concentrations.

TABLE 20
Summary of Plasma Pharmacokinetic Parameters of RO6868847 after
Single Oral Dose Administration under Fasted Conditions

| | Dose | | | | | |
|---|---|---|---|---|---|---|
| | 0.75 mg | 3.0 mg | 10 mg | 30 mg | 100 mg | 300 mg |
| Parameter * | N = 3 | N = 3 | N = 6 | N = 6 | N = 6 | N = 9 |
| Tₘₐₓ | 1.00 | 1.00 | 1.75 | 1.50 | 1.75 | 3.00 |
| (h) | (1.00-3.00) | (1.00-3.00) | (1.00-3.00) | (1.00-3.00) | (1.00-4.00) | (1.50-6.00) |
| Cₘₐₓ | 9.92 | 40.0 | 111 | 355 | 1,120 | 2,500 |
| (ng/mL) | (17.4%) | (5.1%) | (22.7%) | (20.9%) | (18.0%) | (24.1%) |
| AUCᵢₙₜ | 112 | 435 | 972 | 3,530 | 12,500 | 31,400 |
| (ng · h/mL) | (39.3%) | (3.0%) | (19.6%) | (52.9%) | (30.8%) | (28.7%) |
| t_{1/2} | 6.89 | 7.99 | 6.95 | 6.97 | 14.6 | 18.5 |
| (h) | (22.1%) | (4.4%) | (25.1%) | (48.5%) | (57.5%) | (36.6%) |

Median (min, max) for Tₘₐₓ, Geo mean (Geo mean CV %) otherwise

In Part 2 (FE) of Study BP40387, consistent with delayed gastric emptying due to an extended meal, the time to reach peak plasma concentration (Tmax) was slightly longer following dosing after a high fat, high calorie breakfast. Median Tmax was achieved at 4 hours post-dose in the fed state, compared to 3 hours post-dose in the fasted state. On average, Cmax remained unaffected by food and AUCInf increased only marginally in the fed state compared to the fasted state. Single Dose Urine Pharmacokinetics

After single oral dose administration in Part 1 (SAD) of Study BP40387, RO6868847 was quantifiable in urine; however, the fraction of the dose administered (Fe) that was recovered in urine as RO6868847 over 72 hours post-dose was low, with geometric mean values ranging from 2.16% to 4.27%, across the dose range investigated.

### Multiple Dose Plasma Pharmacokinetics

In Part 3 (MAD) of Study BP40387, RO6868847 was rapidly absorbed, with overall individual time to peak plasma concentrations (Tmax) achieved between 1 to 4 hours post-dose in all dose groups (with the exception of the 6 mg dose group on Day 1, for which Tmax ranged from 0.5 to 3 hours) on Days 1, 7, and 14 (Table 21). Table 21 summarizes the plasma PK parameters of RO6868847 at steady-state on Day 14. At the end of the 14-day treatment period, RO6868847 plasma concentrations declined mono-exponentially for the lowest investigated dose of 6 mg RO6868847, however for doses exceeding 6 mg, a bi-exponential decline was observed. The elimination phase was characterized by a geometric mean apparent terminal elimination half-life, ranging from 8.27 hours to 39.0 hours across the explored dose range. The statistical analysis indicated that Cmax and AUCtau at steady-state increased in a slightly less than dose-proportional fashion for doses exceeding 100 mg. However, this effect was more pronounced for Cmax, particularly at a dose of 300 mg RO6868847. Following multiple dose administration steady state was reached approximately after the second day of treatment, with minor accumulation, independent of dose, for both Cmax and AUCtau (Table 21). The statistical analysis of the log-transformed accumulation ratios indicated that there is no evidence of time-dependent pharmacokinetics.

TABLE 21
Summary of Plasma PK Parameters of RO6868847 after Once-Daily Oral Dose
Administration of RO6868847 under Fasted Conditions on Day 14

| | 6.0 mg | 20 mg | 60 mg | 200 mg | 300 mg |
|---|---|---|---|---|---|
| Parameter * | N = 6 | N = 6 | N = 6 | N = 5 | N = 6 |
| Tₘₐₓ (h) | 3.00 | 1.06 | 2.00 | 3.00 | 2.50 |
| | (1.00-3.02)) | (1.00-4.00) | (1.00-3.00) | (1.00-4.00) | (1.50-4.00) |
| Cₘₐₓ | 97.9 | 289 | 899 | 2,380 | 2,970 |
| (ng/mL) | (11.1%) | (37.7%) | (23.0%) | (19.4%) | (16.6%) |
| AUCₜₐᵤ | 870 | 2,610 | 8,480 | 21,400 | 27,100 |
| (ng · h/mL) | (18.3%) | (39.3%) | (25.5%) | (20.9%) | (22.3%) |
| T_{1/2} (h) | 8.27 | 15.0 | 31.6 | ND | 39.0 |
| | (25.4%) | (108.5%) | (39.1%) | | (24.4%) |

* Mediam (min, max) for Tₘₐₓ, Geo mean (Geo mean CV %) otherwise. ND = Not Determined

### Multiple Dose Urine Pharmacokinetics

At steady-state, RO6868847 was quantifiable in urine; however, the Fe that was recovered in urine as RO6868847 over the dosing interval (24 hours) was low, with geometric mean values ranging from 0.738% to 1.85%, across the dose range investigated.

### Safety in Phase 1 BP40387

In Study BP40387, in Part 1, 11 participants (24.4%), Part 2, 1 participant (12.5%), Part 3, 16 participants (40%), Part 4, 12 participants (70.6%) and Part 5, 2 participants (11.1%), had reported at least 1 AE (see Table 22). One participant (5.9%) in Part 4 of the study was withdrawn by the investigator because of AEs. The AEs reported in more than 2 participants in Part 1 was medical device site erythema (6 participants in each PT) and vascular procedure complication (2 participants in each PT), in Part 3 was medical device site erythema (5 participants in each PT), constipation (3 participants in each PT) medical device site papule, medical device site pruritus, vascular procedure complication, headache (each 2 participants), and in Part 4 constipation (2 participants in each PT). In Study BP40387 one SAE was reported in Part 4 (DDI-CYP Induction) of severe intensity (T-wave morphology significantly changed compared to baseline) and moderate intensity (concurrent tachycardia) in a subject treated with 300 mg vicasinabin QD. The event was considered to be related to vicasinabin by the investigator and resolved without sequelae within one week after treatment discontinuation.

### Example 3

In example 2, target engagement of RO6868847 was assessed on circulating B-cells from whole blood samples utilizing a newly developed exploratory flow cytometry assay to monitor RO6868847-induced CB2 receptor internalization on B-cells. FIG. 5 displays the mean CB2 receptor target engagement versus time profiles following multiple dose administration of RO6868847 in example 3 excluding data for the 6 mg dose, since it was not reportable. Following multiple dose administration up to 300 mg RO6868847 as described in example 2, a reversible, dose-dependent and sustained CB2 receptor target engagement was observed. Upon treatment discontinuation CB2 receptor surface expression returned to baseline approximately within 1 week. Target engagement appeared to reach a plateau at doses of 100 mg RO6868847 with only marginal increases at higher doses of RO6868847. In example 2, pro-inflammatory cytokine production was induced by ex vivo stimulation with LPS in freshly obtained whole blood samples, to measure downstream markers of RO6868847-mediated CB2 receptor internalization and explore its pharmacological profile. Serum levels of TNF-α, G-CSF, IL-1β, and Interferon-7, of participants in Study BP40387 were assessed to investigate the changes of these cytokines in the presence of repeated dose administration of up to 300 mg RO6868847 once-daily for 14 days. Under steady-state conditions, a concentration-dependent effect for TNF-α, G-CSF, and IL1-β could be observed compared to baseline, as shown in FIG. 6, FIG. 7, and FIG. 8, respectively. However, IFN-γ did not show any detectable trend within the explored concentration range of RO6868847 (see FIG. 9). It should be noted that the IL1-β analysis was negatively affected by a substantial proportion of baseline data, not being reportable (see FIG. 8), especially at the highest tested doses of 300 mg of RO6868847. Substantial variability in these measurements was observed. However, this was expected and no dedicated predefined statistical analysis was performed since cohort sizes were small for this exploratory assessment.

### CONCLUSIONS

### Safety and Tolerability

RO6868847 was safe and very well tolerated in healthy male and female participants following single oral doses up to 300 mg.
RO6868847 was generally well tolerated in healthy male and female participants following multiple oral doses administered once daily for 14 days up to 300 mg of RO6868847, with the sole exception of one participant who developed a SAE which's onset coincided, with a transiently increased plasma trough concentration, of unknown reason(s), of approximately 4- to 5-fold above the average of the remaining participants of the cohort at the dose of 300 mg RO6868847.
There was no dose-related increase in the incidence or severity of reported AEs following single and multiple dose administration of RO6868847 and no cluster of adverse events was observed.
No specific concerns were identified regarding clinical laboratory parameters, ECGs, vital signs, C-SSRS, Bowdle VAS, Bond and Lader VAS, TBNK assay, or neurological examinations.

### Pharmacokinetics and Pharmacodynamics

RO6868847 was rapidly absorbed, and following single and multiple oral dose administration, no pronounced deviations from dose-proportionality were observed for AUC and Cmax up to a dose of 100 mg RO6868847. However, for doses beyond 100 mg RO6868847 AUC and Cmax appeared to increase in a slightly less than dose-proportional fashion.
Following multiple dose administration of RO6868847 once-daily, steady state was on average achieved approximately after the second day of dosing with only minor accumulation.
Overall, food had no relevant effect on pharmacokinetics of RO6868847.
Renal excretion of RO6868847 following single and multiple dose administration was minor, accounting on average for less than 5% of the dose administered.
Administration of single and multiple doses of RO6868847 resulted in a dose-dependent, long-lasting, but reversible CB2 receptor internalization on B cells in blood.
Multiple dose administration of RO6868847 appeared to attenuate ex vivo LPS induced pro-inflammatory cytokine production (i.e., TNF-α, G-CSF, and IL1-β) in whole blood compared to baseline in an overall dose-dependent fashion.

### Aspects of the Present Invention

Aspect 1. The compound (3S)-1-[5-tert-butyl-3-[(1-methyltetrazol-5-yl)methyl]triazolo[4,5-d]pyrimidin-7-yl]pyrrolidin-3-ol for use as agonist of the Cannabinoid Receptor 2, at a dose between 0.75 mg and 300 mg in a patient.
Aspect 2. The compound according to aspect 1, for use in the treatment or prevention of chronic degenerative diseases.
Aspect 3. The compound according to aspects 1 or 2, for use in the treatment or prevention of neurodegenerative inflammatory disorders, atherosclerosis, liver fibrosis, or microvascular diabetic complications.
Aspect 4. The compound according to any one of aspects 1 to 3, for use in the treatment or prevention of microvascular diabetic complications, wherein microvascular diabetic complications are diabetic retinopathy, diabetic macular edema, or non-proliferative diabetic retinopathy.
Aspect 5. The compound according to any one of aspects 1 to 4, for use in the treatment or prevention of non-proliferative diabetic retinopathy (NPDR).
Aspect 6. The compound according to any one of aspects 1 to 5, particularly wherein the patient is a human (such as a male or female human).
Aspect 7. The compound according to any one of aspects 1 to 6, wherein it is orally administered.
Aspect 8. The compound according to any one of aspects 1 to 7, wherein the patient is a human (such as a male or female human), wherein the compound is being administered, in particular administered orally, at a dose between 0.75 mg and 300 mg.
Aspect 9. The compound according to any one of aspects 1 to 8, wherein it is administered once a day.
Aspect 10. The compound according to any one of aspects 1 to 9, wherein it is administered in combination with other treatments, in particular in combination with laser, anti-VEGF treatments, or other treatments for diabetic retinopathy.
Aspect 11. A method for the treatment of an agonist of the Cannabinoid Receptor 2, comprising administering to a patient (in particular a patient in need thereof), (3S)-1-[5-tert-butyl-3-[(1-methyltetrazol-5-yl)methyl]triazolo[4,5-d]pyrimidin-7-yl]pyrrolidin-3-ol at a dose between 0.75 mg and 300 mg.
Aspect 12. The method according to aspect 11, for the treatment or prevention of chronic degenerative diseases.
Aspect 13. The method according to aspect 11 or 12, for the treatment or prevention of neurodegenerative inflammatory disorders, atherosclerosis, liver fibrosis, or microvascular diabetic complications.
Aspect 14. The method according to any one of aspects 11 to 13, for the treatment or prevention of microvascular diabetic complications, wherein microvascular diabetic complications are diabetic retinopathy, diabetic macular edema, or non-proliferative diabetic retinopathy.
Aspect 15. The method according to any one of aspects 11 to 14, for the treatment or prevention of non-proliferative diabetic retinopathy (NPDR).
Aspect 16. The method according to any one of aspects 11 to 15, particularly wherein the patient is a human (such as a male or female human).
Aspect 17. The method according to any one of aspects 11 to 16, wherein it is orally administered.
Aspect 18. The method according to any one of aspects 11 to 17, wherein the patient is a human (such as a male or female human), wherein the compound is being administered, in particular administered orally, at a dose between 0.75 mg and 300 mg.
Aspect 19. The method according to any one of aspects 11 to 18, wherein it is administered once a day.
Aspect 20. The method according to any one of aspects 11 to 19, wherein it is administered in combination with other treatments, in particular in combination with laser, anti-VEGF treatments, or other treatments for diabetic retinopathy.
Aspect 21. The use of compound (3S)-1-[5-tert-butyl-3-[(1-methyltetrazol-5-yl)methyl]triazolo[4,5-d]pyrimidin-7-yl]pyrrolidin-3-ol in the manufacture of a medicament as agonist of the Cannabinoid Receptor 2 at dose between 0.75 mg and 300 mg.
Aspect 22. The use of aspect 21, for the manufacture of a medicament for the treatment or prevention of chronic degenerative diseases.
Aspect 23. The use of aspect 21 or 22, for the manufacture of a medicament for the treatment or prevention of neurodegenerative inflammatory disorders, atherosclerosis, liver fibrosis, or microvascular diabetic complications.
Aspect 24. The use of any one of aspects 21 to 23, for the manufacture of a medicament for the treatment or prevention of microvascular diabetic complications, wherein microvascular diabetic complications are diabetic retinopathy, diabetic macular edema, or non-proliferative diabetic retinopathy.
Aspect 25. The use of any one of aspects 21 to 24, for the manufacture of a medicament for the treatment or prevention of non-proliferative diabetic retinopathy (NPDR).
Aspect 26. The use of any one of aspects 21 to 25, particularly wherein the patient is a human (such as a male or female human).
Aspect 27. The use of any one of aspects 21 to 26, wherein it is orally administered.
Aspect 28. The use of any one of aspects 21 to 27, wherein the patient is a human (such as a male or female human), wherein the compound is being administered, in particular administered orally, at a dose between 0.75 mg and 300 mg.
Aspect 29. The use of any one of aspects 21 to 28, wherein it is administered once a day.
Aspect 30. The use of any one of aspects 21 to 28, wherein it is administered in combination with other treatments, in particular in combination with laser, anti-VEGF treatments, or other treatments for diabetic retinopathy.

1.-10. (canceled)

11. A method for the treatment of an agonist of the Cannabinoid Receptor 2, comprising administering to a patient (3S)-1-[5-tert-butyl-3-[(1-methyltetrazol-5-yl)methyl]triazolo[4,5-d]pyrimidin-7-yl]pyrrolidin-3-ol at a dose between 0.75 mg and 300 mg.

12. The method according to claim 11, for the treatment or prevention of chronic degenerative diseases.

13. The method according to claim 11, for the treatment or prevention of neurodegenerative inflammatory disorders, atherosclerosis, liver fibrosis, or microvascular diabetic complications.

14. The method according to claim 11, for the treatment or prevention of microvascular diabetic complications, wherein microvascular diabetic complications are diabetic retinopathy, diabetic macular edema, or non-proliferative diabetic retinopathy.

15. The method according to claim 11, for the treatment or prevention of non-proliferative diabetic retinopathy (NPDR).

16. The method according to claim 11, particularly wherein the patient is a human.

17. The method according to claim 11, wherein (3S)-1-[5-tert-butyl-3-[(1-methyltetrazol-5-yl)methyl]triazolo[4,5-d]pyrimidin-7-yl]pyrrolidin-3-ol is orally administered.

18. The method according to claim 11, wherein the patient is a human wherein (3S)-1-[5-tert-butyl-3-[(1-methyltetrazol-5-yl)methyl]triazolo[4,5-d]pyrimidin-7-yl]pyrrolidin-3-ol is administered orally, at a dose between 0.75 mg and 300 mg.

19. The method according to claim 11, wherein (3S)-1-[5-tert-butyl-3-[(1-methyltetrazol-5-yl)methyl]triazolo[4,5-d]pyrimidin-7-yl]pyrrolidin-3-ol is administered once a day.

20. The method according to claim 11, wherein (3S)-1-[5-tert-butyl-3-[(1-methyltetrazol-5-yl)methyl]triazolo[4,5-d]pyrimidin-7-yl]pyrrolidin-3-ol is administered in combination with laser, an anti-VEGF treatment, or another treatment for diabetic retinopathy.

21.-31. (canceled)
